(19) United States
(12) Patent Application Publication
(10) Pub. No.: US 2011/0152631 A1
(43) Pub. Date: Jun. 23, 2011

(54) MEDICAL DIAGNOSTIC APPARATUS AND METHOD OF OPERATING THE SAME

(75) Inventors: Su Myeong Lee, Gyeonggi-do (KR); Yong Ho Lee, Seoul (KR)

(73) Assignee: MADISON CO., LTD.

(21) Appl. No.: 12/971,755

(22) Filed: Dec. 17, 2010

(30) Foreign Application Priority Data
Dec. 17, 2009 (KR) 10-2009-0126080

(51) Int. Cl. A61B 5/00

(52) U.S. Cl. 600/300

(57) ABSTRACT

The present disclosure relates to a medical diagnostic apparatus and a method of operating the same. The medical diagnosis apparatus performs measurement based on a preset diagnosis system environment in response to a measurement start instruction input from a user, and only if the apparatus receives an instruction for changing the diagnosis system environment, the apparatus performs measurement based on a changed diagnosis system environment in response to the instruction. In the apparatus and method, a workflow is arranged to perform measurement accurately, efficiently and safely, thereby reducing time for measurement or diagnosis and decreasing the occurrence of user error.

### TECHNICAL FIELD

The present subject matter relates to equipment and techniques to allow effective medical diagnostic measurements that reduce measurement and diagnosis time, increase user and patient convenience and reduce user error.

### BACKGROUND

Generally, a medical diagnostic apparatus such as an ultrasonic diagnostic apparatus has various functions to provide diagnostic medical measurements for a patient. These functions may be performed independently or cooperatively. The manner in which these functions are performed by medical diagnostic apparatuses is referred to as a workflow. For example conventional medical diagnostic apparatuses repeat routine operations prior to performing the actual diagnostic measurement, which may result in long delays in performing the actual diagnostic measurement, increased chance of user/operator error, prolonged apparatus use time and inconvenience to the patient due to the long time needed to obtain measurements.

The workflow of the above described conventional medical diagnosis apparatus, such as an ultrasonic diagnostic apparatus is shown, in FIG. 1. As shown in FIG. 1. a user first selects a probe in S1 and then selects a diagnosis application in S2. As used herein, the term “probe” refers to any part of a medical diagnostic apparatus that is used to obtain diagnostic information from patient. Examples of such probes may include ultrasound transducers, thermometers and catheters. As used herein, the term “diagnosis application” refers to a certain medical department or field, such as obstetrics, genecology, gastroenterology and the like.

Step S3 of FIG. 1 requires confirmation of whether a change of an operation mode is needed. If a change of the operation mode is needed, the operation mode is changed in response to a user's selection in S4 and various types of parameters related to a diagnosis image are also adjusted in S5. Then, the apparatus receives a measurement start instruction from the user in S6. If it is confirmed in S3 that the change of the operation mode is not needed, the process directly proceeds to S5. Here, the term “operation mode” refers to a variety of modes related to a diagnosis image of the medical diagnosis apparatus, for example, a two-dimensional (2D) display mode, three-dimensional (3D) display mode, Doppler mode, color mode, and the like. Further, the parameters may include a variety of parameters related to the diagnosis image of the medical diagnosis apparatus, for example, a scale, zoom, focus, time gain compensation (TGC), gain, and the like.

Next, it is confirmed in S7 whether a measurement item to be currently performed is a target measurement item. If the measurement item to be currently performed is the target measurement item, the apparatus receives an item selection signal input from the user in S9. If it is confirmed in S7 that the target measurement item to be currently performed is not the target measurement item, the user shifts to the next item in S8 and performs the confirmation operation again in S7. Here, the term “target measurement item” may mean a specific portion of an object to be measured by the medical diagnostic apparatus, or a specific measurement item of the specific portion. For example, when a fetus is subjected to ultrasound diagnosis, the target measurement item means a specific portion, such as the head, the legs, the stomach, the womb of the mother, or the like, for each group, such as an initial fetus, a general fetus, or the like, or means a specific target measurement item of the specific portion, such as a diameter of the head of a fetus.

Furthermore, if the target measurement item is selected in S9, the measurement is performed in S10. Then, it is confirmed in S11 whether the user wants to continue the measurement. If the user wants to continue the measurement, the process returns back to the operation in S7 and repeats the above operations. Here, the confirmation of whether the user wants to continue the measurement is to confirm whether the user wants to continue the measurement under diagnosis conditions set in S1˜S5.

In addition, if it is confirmed in S11 that the user does not want to continue the measurement, it is confirmed in S12 whether the user wants to annotate a measurement result, i.e. provide informational notes regarding the measurement. If the user wants to annotate the measurement result, the annotation is provided to the result in S13, and the content of annotation is stored in storage in S14, after the annotation operation is finished. If the user does not want to annotate the result, the process proceeds to S15 described below. Herein, the annotation includes a body marker as well as a general annotation.

Finally, it is confirmed whether the user wants to continue the measurement, and if the answer is yes, the process returns back to S1 and repeats the above operations. If the answer is no, the measurement is finished in S15. This multi-step procedure may result in several drawbacks. For instance, although the main purpose of the medical diagnosis apparatus such as an ultrasonic diagnostic apparatus is to obtain an image of a diagnosis object and a diagnosis result based on measurement of the image, a user is required to perform a series of preliminary operations prior to the actual measurement. Such lengthy preliminary procedures may make it difficult for the user to focus on performing the actual measurement, thereby decreasing operation efficiency.

Furthermore, even in the case where the measurement is repetitiously performed according to the same workflow, conventional apparatuses require a user to inconveniently repeat a series of operations, for example, searching for or moving to a target measurement item and then selecting the target measurement item. Therefore, this approach may result in decreased operation efficiency, and increased user error.

Hence a need exists for a medical diagnostic apparatus that provides accurate diagnostic measurements. Furthermore, there is a need for a method of operating a medical diagnostic apparatus that is safe and efficient.

### SUMMARY

To improve over the art and address one or more of the needs outlined above, diagnostic medical apparatuses are used to provide efficient diagnostic measurements.

In one general aspect, the instant application describes a method of operating a medical diagnostic apparatus, wherein the medical diagnostic apparatus performs a first measurement based on a preset diagnosis system environment in response to a measurement start instruction input from a user. In addition, only if the apparatus receives an instruction for changing the diagnosis system environment, does the apparatus perform a second measurement based on this changed diagnosis system environment in response to the instruction.

The above general concept may include one or more of the following features. For example, the method may further include steps of receiving the measurement start instruction; confirming whether a change of the preset diagnosis system environment is needed; and performing the second measurement. Thus, the second measurement is performed by reflecting the change in diagnosis system environment only upon confirming that the change of the preset diagnosis system environment is needed.

Furthermore, the diagnosis system environment may include information about at least one of a probe, a diagnosis application, an operation mode, and at least one parameter related to the diagnostic measurement.

The step of confirming whether a change of the preset diagnosis system environment is needed may include: confirming whether a change of a diagnosis application is needed; and confirming whether a change of an operation mode is needed. The step of confirming whether a change of operation mode is needed may be performed after changing the diagnosis application only upon confirming that the change of the diagnosis application is needed.

In addition, the step of confirming whether a change of the preset diagnosis system environment is needed may include confirming whether a change of an operation mode is needed; and confirming whether a change of a diagnosis application is needed. The step of confirming whether a change of diagnosis application is needed may be performed after changing the operation mode only upon confirming that the change of the operation mode is needed.

The step of confirming whether a change of the present diagnosis system environment is needed may further include confirming whether a change of a probe is needed. The step of confirming whether a change of a probe is needed may be provided before the step of confirming whether a change of an operation mode is needed, after the step of confirming whether a change of an operation mode is needed, or after the step of confirming whether a change of a diagnosis application is needed, wherein the probe is changed to proceed to the next step, only upon confirming that the change of the probe is needed.

The step of confirming whether a change of the present diagnosis system environment is needed may further include confirming whether a change of a parameter is needed. The step of confirming whether a change of a parameter is needed may be provided before the step of confirming whether a change of an operation mode is needed, after the step of confirming whether a change of an operation mode is needed, or after the step of confirming whether a change of a diagnosis application is needed, The parameter is changed to proceed to the next step, only upon confirming that the change of the parameter is needed.

The step of confirming whether a change of the present diagnosis system environment is needed may further include confirming whether a change of a probe is needed; and confirming whether a change of a parameter is needed. These steps of confirming whether a change of a probe or a parameter is needed may each be performed at any selective time point.

The method may further include confirming whether a user wants to continue to operate the medical diagnosis apparatus after the step of performing the measurement; and returning to the step of confirming whether a change of the preset diagnosis system environment is needed, upon confirming that the user wants to continue to operate the medical diagnosis apparatus.

Furthermore, the method may further include annotating a measurement result in response to a selection of the user between the step of performing the measurement and the step of confirming whether the user wants to continue to operate the medical diagnosis apparatus.

The at least one parameter may include at least one of a scale, zoom, focus, time gain compensation (TGC), and gain.

The preset diagnosis system environment may be set by a user using an editing function.

In accordance with another general aspect, the present application describes a medical diagnostic apparatus including: an input unit receiving an input from a user; a storage storing information about a preset diagnosis system environment; a controller controlling the apparatus to perform a first measurement based on the preset diagnosis system environment; and an output unit. The controller controls the medical diagnosis apparatus to perform the first measurement based on the preset diagnosis system environment in response to a measurement start instruction input from the user, and only if the apparatus receives an instruction for changing the diagnosis system environment, does the controller allow the apparatus to perform the second measurement based on a changed diagnosis system environment in response to the instruction.

The controller may confirm whether a change of the preset diagnosis system environment is needed, in response to the measurement start instruction input, and control the apparatus to perform the first measurement if the change of the preset diagnosis system condition is not needed, while allowing the apparatus to perform the second measurement by reflecting the change in diagnosis system environment only if the change of the preset diagnosis system environment is needed.

The diagnosis system environment may include information about at least one of a probe, a diagnosis application, an operation mode, and at least one parameter.

When changing the preset diagnosis system environment, the apparatus may change at least one of the probe, the diagnosis application, the operation mode, and the at least one parameter.

The apparatus may further include an editing unit for editing or changing the preset diagnosis system environment.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1 illustrates a flowchart of a method of operating a conventional medical diagnosis apparatus;

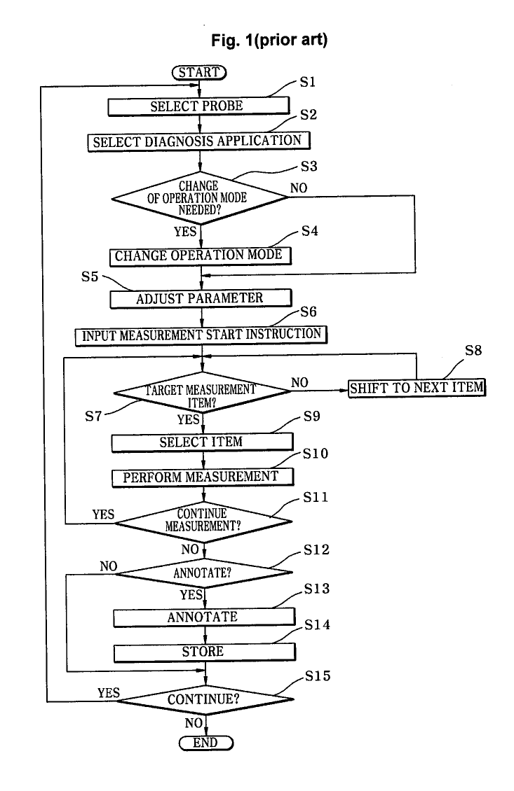

FIG. 2 is a simplified functional block diagram of an exemplary medical diagnosis apparatus.

FIG. 3 illustrates a flowchart of an exemplary method of operating an exemplary medical diagnostic apparatus.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures, components, have been described at a relatively high-level, without detail, in order to avoid unnecessarily obscuring aspects of the present teachings.

The various technologies disclosed herein relate to a method of operating a medical diagnostic apparatus. The teachings herein alleviate one or more of the above noted problems with using a mobile device to conduct transactions.

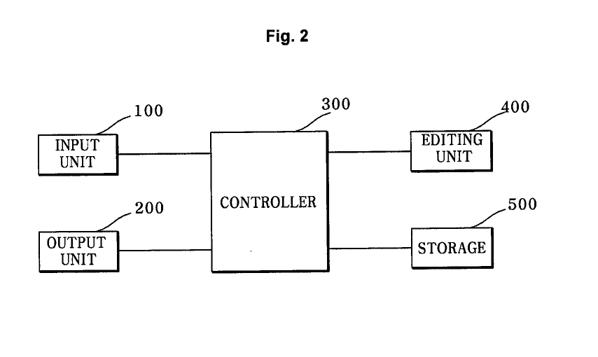

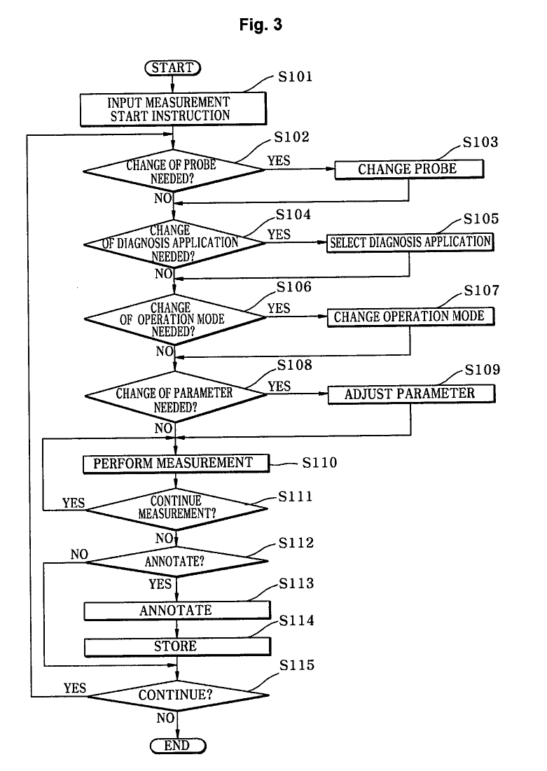

FIG. 2 is a simplified functional block diagram of an exemplary medical diagnostic apparatus, and FIG. 3 is a flowchart of an exemplary method of operating an exemplary medical diagnosis apparatus.

Referring to FIG. 2, the medical diagnostic apparatus includes an input unit 100 receiving an input from a user; storage 500 storing information about a preset diagnosis system environment; a controller 300 controlling the apparatus to perform measurement based on the preset diagnosis system environment; an output unit 200; and an editing unit 400 allowing the user to edit or change the preset diagnosis system environment. For example, the input unit may be a keyboard, tablet, touch-screen or any device that allows user input.

The storage may be any machine readable type media, including any or all of the tangible memory of the computers, processors or the like, or associated modules thereof, such as various semiconductor memories, tape drives, disk drives and the like, which may provide non-transitory storage at any time. Hence, a machine readable medium may take many forms, including but not limited to, a tangible storage medium, a carrier wave medium or physical transmission medium.

Non-volatile storage media include, for example, optical or magnetic disks, such as any of the storage devices in any computer(s) or the like, such as may be used to implement the to automatically providing directions on a mobile station of a customer at a first store to the location of a second store that has a desired product not available in the first store as shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution.

The controllers may be a computer or any other device having a central processing unit (CPU), in the form of one or more processors, for executing program instructions stored on a machine readable medium. The output unit may be a monitor, screen, print-out or other viewable medium.

In the medical diagnostic apparatus, information of a diagnosis system environment is preset and stored in the storage 500. Here, the information of the diagnosis system environment may be an initial preset value originally stored in the medical diagnostic apparatus. Further, the information of the diagnosis system environment may be newly set by a user using the editing unit 400 or may be reset by editing or changing a certain preset value using the editing unit 400. The diagnosis system environment may include a diagnosis application, an operation mode, at least one measurement parameter, and other preset conditions, which may be set to perform measurement in the medical diagnosis apparatus. For example, for an ultrasonic diagnostic apparatus, the diagnosis system environment may include information about probes. The diagnosis application, operation mode and measurement parameters are the same as those disclosed above.

An exemplary method of operating the medical diagnostic apparatus is described with reference to the workflow flow chart illustrated in FIG. 3.

Referring to FIG. 3, the medical diagnostic apparatus receives a measurement start instruction input from a user in S101, via the input unit 100 and it is confirmed in S102 whether a change of a probe is needed. As discussed above, the term probe as used herein refers to any part of a medical diagnostic apparatus that is used to obtain diagnostic information from patients. Examples of such probes may include ultrasound transducers, thermometers and catheters.

That is, the measurement start instruction is first input in order to achieve satisfactory measurement.

If it is confirmed in S102 that the change of the probe is not needed, the process proceeds to S104. Only if the change of the probe is needed, the probe is changed to a newly selected probe in S103. Herein, the operations in S102 and S103 related to change or selection of the probe may also be applied to an ultrasonic diagnostic apparatus and may be omitted depending on the kind of medical diagnosis apparatus.

Then, it is confirmed in S104 whether a change of a diagnosis application is needed. As used herein, the term “diagnosis application” refers to a certain medical department or field, such as obstetrics, genecology, gastroenterology and the like. If it is confirmed in S104 that the change of the diagnosis application is not needed, the process proceeds to S106. Only if the change of the diagnosis application is needed, the diagnosis application is changed to a newly selected diagnosis application in S105.

Then, it is confirmed in S106 whether a change of an operation mode is needed. As noted above, the term “operation mode” as used herein refers to a variety of modes related to a diagnosis image of the medical diagnosis apparatus, for example, a two-dimensional (2D) display mode, three-dimensional (3D) display mode, Doppler mode, color mode, and the like. If it is confirmed in S106 that the change of the operation mode is not needed, the process proceeds to S108. Only if the change of the operation mode is needed, the operation mode is changed to a newly selected operation mode in S107.

Then, it is confirmed in S108 whether a change of a parameter is needed. As discussed above, parameters may include a variety of parameters related to the diagnosis image of the medical diagnosis apparatus, for example, a scale, zoom, focus, time gain compensation (TGC), gain, and the like.

If it is confirmed that the change of the parameter is not needed, the process proceeds to the next operation to perform measurement in S110. Only if the change of the parameter is needed, parameters related to various kinds of diagnosis images are modified or adjusted in S109 and the measurement is performed in S110.

As such, in the present medical diagnosis apparatus confirms, through the operations in S102, S104, S106 and S108, whether a change of the preset diagnosis system environment including at least one of the probe, diagnosis application, operation mode and at least one parameter is needed. The medical diagnosis apparatus directly performs the measurement if it is confirmed that the change of the preset diagnosis system environment is not needed, so that the number of unnecessary operations is minimized in performing the measurement, thereby reducing required operating time and operating frequency of the user required for an actual measurement or diagnosis and providing convenience in operation.

Next, it is confirmed in S111 whether a user wants to continue the measurement. If the answer is yes, the process returns back to the operation in S110 to perform the measurement, and if the answer is no, the process proceeds to S112. Here, the confirmation of whether the user wants to continue the measurement is to confirm whether the user wants to continue the measurement under the overall diagnosis system environment preset in the current stage. When the process returns back to the operation in S110 to continue the measurement, the method may further include searching for a specific measurement item to allow a user to select the specific measurement item as a desired one, if the user wants to change a current measurement item to the specific measurement item to be performed by the diagnostic apparatus.

If it is confirmed in S111 that the user does not want to continue the measurement, it is confirmed in S112 whether the user wants to annotate a measurement result. If it is confirmed that the user wants to annotate the result, the annotation is provided to the result in S113 and the content of annotation is stored in the storage 500 after the annotation operation is finished, in S114. If it is confirmed that the user does not want to annotate the result, the process proceeds to step S115 described below.

Next, if it is confirmed that the user wants to continue the measurement, the process returns back to S102 and repeats the above operations. If the user does not want to continue the measurement, the measurement is finished in S115. Here, according to another embodiment, the process may return back from the operation in S115 to any one of the operations in S104, S106 and S108 instead of returning back to the operation in S102.

Alternatively, the operations in S102, S104 S106 and S108 may be performed in a different sequence from that shown in FIG. 3. For example, the operations in S106 and S108 may be performed before the operations in S102 and S104. Further, at least one of the operations in S102, S104 S106 and S108 may be selectively applied.

As such, in the medical diagnostic apparatus and method of operating the same described in the present application, a workflow is arranged to effectively perform the primary purpose of the medical diagnosis apparatus—that of effectively making a diagnostic measurement, such that general operations except for the measurement may be automatically set and performed based on a preset diagnosis system environment and may be changed to reflect a change or modification of the diagnosis system environment if needed. In this manner, the apparatus and method described in the present application significantly reduces the time for measurement or diagnosis and reduces the incidence of operation failure by providing an efficient, accurate and convenient method of operation.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

What is claimed is:

1. A method of operating a medical diagnostic apparatus, wherein the medical diagnostic apparatus performs a first measurement based on a preset diagnosis system environment in response to a measurement start instruction input from a user, and wherein only if the apparatus receives an instruction for changing the diagnosis system environment, the apparatus performs a second measurement based on a changed diagnosis system environment in response to the instruction.

2. The method of claim 1, comprising:
1) receiving the measurement start instruction;
2) confirming whether a change of the preset diagnosis system environment is needed; and
3) performing the first measurement,
wherein the second measurement is performed by reflecting the change in diagnosis system environment only upon confirming that the change of the preset diagnosis system environment is needed.

3. The method of claim 2, wherein the diagnosis system environment comprises information about at least one of a probe, a diagnosis application, an operation mode, and at least one parameter.

4. The method of claim 3, wherein the step 2) of confirming whether the change of the preset diagnosis system environment is needed comprises:
a) confirming whether a change of a diagnosis application is needed; and
b) confirming whether a change of an operation mode is needed,
the step b) being performed after changing the diagnosis application only upon confirming that the change of the diagnosis application is needed.

5. The method of claim 3, wherein the step 2) of confirming whether the change of the preset diagnosis system environment is needed comprises:
a) confirming whether a change of an operation mode is needed; and
b) confirming whether a change of a diagnosis application is needed,
the step b) being performed after changing the operation mode only upon confirming that the change of the operation mode is needed.

6. The method of claim 4, wherein the step 2) of confirming whether the change of the preset diagnosis system environment is needed further comprises:
c) confirming whether a change of a probe is needed, before the step a), after the step a), or after the step b),
the probe being changed to proceed to the next step only upon confirming that the change of the probe is needed.

7. The method of claim 4, wherein the step 2) of confirming whether the change of the preset diagnosis system environment is needed further comprises optionally:
d) confirming whether a change of a parameter is needed, before the step a), after the step a), or after the step b),
the parameter being changed to proceed to the next step only upon confirming that the change of the parameter is needed.

8. The method of claim 4, wherein the step 2) of confirming whether the change of the preset diagnosis system environment is needed further comprises:
c) confirming whether a change of a probe is needed; and
d) confirming whether a change of a parameter is needed,
wherein the steps c) and d) are performed at any selective time point.

9. The method of claim 4, further comprising:
confirming whether a user wants to continue to operate the medical diagnosis apparatus after the step 3) of performing the measurement; and
returning to the step 2) of confirming whether a change of the preset diagnosis system environment is needed, upon confirming that the user wants to continue to operate the medical diagnosis apparatus.

10. The method of claim 9, further comprising:
annotating a measurement result in response to a selection of the user between the step 3) of performing measurement and the step 4) of confirming whether the user wants to continue to operate the medical diagnosis apparatus.

11. The method of claim 3, wherein the at least one parameter comprises at least one selected from the group consisting of a scale, zoom, focus, time gain compensation (TGC), and gain.

12. The method of claim 1, wherein the preset diagnosis system environment is set by a user using an editing function.

13. A medical diagnostic apparatus comprising:
an input unit receiving an input from a user;
a storage for storing information about a preset diagnosis system environment;
a controller for controlling the apparatus to perform a first measurement based on the preset diagnosis system environment; and
an output unit,
wherein the controller controls the medical diagnosis apparatus to perform the measurement based on the preset diagnosis system environment in response to a measurement start instruction input from the user, and
wherein only if the apparatus receives an instruction for changing the diagnosis system environment, the controller allows the apparatus to perform a second measurement based on a changed diagnosis system environment in response to the instruction.

14. The medical diagnostic apparatus of claim 13, wherein the controller confirms whether a change of the preset diagnosis system environment is needed, in response to the measurement start instruction input, and controls the apparatus to perform the first measurement if the change of the preset diagnosis system condition is not needed, while allowing the apparatus to perform the second measurement by reflecting the change in diagnosis system environment only if the change of the preset diagnosis system environment is needed.

15. The medical diagnostic apparatus of claim 14, wherein the diagnosis system environment comprises information about at least one of a probe, a diagnosis application, an operation mode, and at least one parameter.

16. The medical diagnostic apparatus of claim 15, wherein, when changing the preset diagnosis system environment, the apparatus changes at least one of the probe, the diagnosis application, the operation mode, and the at least one parameter.

17. The medical diagnostic apparatus of claim 13, further comprising: an editing unit for editing or changing the preset diagnosis system environment.
